(19) United States
(12) Patent Application Publication
(10) Pub. No.: US 2007/0072816 A1
(43) Pub. Date: Mar. 29, 2007

(54) Methods for inhibiting angiogenesis

(75) Inventors: Yihai Cao, Stockholm (SE); Renhai Cao, Stockholm (SE); Robert Pawliuk, Medford, MA (US); Philippe Leboulch, Charlestown, MA (US)

Correspondence Address: LAHIVE & COCKFIELD, LLP, ONE POST OFFICE SQUARE, BOSTON, MA, 02109-2127, US

(73) Assignee: Genetix Pharmaceuticals, Inc., Cambridge, MA (US)

(21) Appl. No.: 11/403,040

(22) Filed: Apr. 11, 2006

Related U.S. Application Data
(63) Continuation of application No. 10/346,589, filed on Jan. 17, 2003, now abandoned.
(60) Provisional application No. 60/350,005, filed on Jan. 17, 2002.

(51) Int. Cl. A61K 48/00; C12N 15/09

(52) U.S. Cl. 514/44; 435/455

(57) ABSTRACT

Methods for inhibiting angiogenesis using gene therapy are disclosed. Genes encoding PLGF or VEGF-B are delivered to cells e.g., tumor cells, which express VEGF, such that heterodimers of PLGF/VEGF and/or VEGF-B/VEGF are formed within the cells, preferably at a greater ratio than homodimers of VEGF/VEGF. The heterodimers have reduced angiogenic activity compared to VEGF homodimers.

### RELATED APPLICATIONS

This application is a continuation of U.S. patent application Ser. No. 10/346,589, filed on Jan. 17, 2003, which claims priority to U.S. provisional patent application Ser. No. 60/350,005, filed on Jan. 17, 2002, the entire contents of which are incorporated herein by this reference.

### BACKGROUND OF INVENTION

Tumor growth and metastasis are dependent on the degree of neovascularization in the tumor bed (Carmeliet, P et al., (2000) Nature 407, 249-57; Folkman, J (1995) Nat Med 1, 27-31; Hanahan, D. & Folkman, J. Cell (1996) 86, 353-64). Vascular endothelial growth factor (VEGF) is a key angiogenic factor that is frequently utilized by tumors and other tissues to switch on blood vessel growth (Dvorak, H. F. (2000) Semin Perinatol 24, 75-8; Ferrara, N. & Alitalo, K (1999) Nat Med 5, 1359-64; Yancopoulos, G. D. et al., (2000) Nature 407, 242-8; Benjamin, L. E. & Keshet, E. (1997) Proc Natl Acad Sci USA 94, 8761-6). VEGF also increases vascular permeability, which is important for tumor invasion and metastasis (Dvorak, H. F et al., (1999) Curr Top Microbiol Immunol 237, 97-132; Senger, D. R. et al., (1983) Science 219, 983-5). In addition to pathological angiogenesis, VEGF is an essential factor that contributes to the development of the vascular system by stimulating vasculogenesis and angiogenesis during the embryonic development (Carmeliet, P. et al., (1996) Nature 380, 435-9; Ferrara, N. et al., (1996) Nature 380, 439-42).

The VEGF family is comprised of six structurally related members that include VEGF, the prototype of VEGF, placenta growth factor (PLGF), VEGF-B, VEGF-C, VEGF-D and VEGF-E (Eriksson, U. & Alitalo, K. (1999) Curr Top Microbiol Immunol 237, 41-57). The biological functions of the VEGF family are mediated by activation of at least three structurally homologous tyrosine kinase receptors, VEGFR-1/Flt-1, VEGFR-2/Flk-1/KDR and VEGFR-3/Flt-4 (Cao, Y. et al., (1998) Proc Natl Acad Sci USA 95, 14389-94). VEGF and PLGF also bind to a non-tyrosine kinase receptor neuropilin-1 (Migdal, M. et al., (1998). J Biol Chem 273, 22272-8; Soker, S et al., 1998) Cell 92, 735-45). According to their receptor binding patterns and angiogenic features, the VEGF family can be further divided into three subgroups: 1) VEGF, which binds to VEGFR-1 and VEGFR-2, and induces vasculogenesis, angiogenesis and vascular permeability; 2) PLGF and VEGF-B, which bind only to VEGFR-1, and their physiological and pathological roles remain unknown; and 3) VEGF-C and VEGF-D, which interact with both VEGFR-2 and VEGFR-3, and induce both blood angiogenesis and lymphangiogenesis (Cao, Y. et al supra; Makinen, T. et al., (2001) Nat Med 7, 199-205; Marconcini, L. et al., (1999) Proc Natl Acad Sci USA 96, 9671-6; Skobe, M. et al., (2001) Nat Med 7, 192-8; Stacker, S. A. et al., (2001) Nat Med 7, 186-91). Accumulating evidence has suggested to that VEGFR-2, in response to VEGF, mediates angiogenic signals for blood vessel growth and VEGFR-3 transduces signals for lymphatic vessel growth (Dvorak, H. F. supra; Ferrara, N. & Alitalo, K. supra; Ferrara, N. (1999) Curr Top Microbiol Immunol 237, 1-30).

Similar to the platelet growth factor (PDGF) family, all members in the VEGF family naturally exist as dimeric forms in order to interact with their specific receptors. In addition to homodimers, PLGF and VEGF-B can form heterodimers with VEGF when these factors are produced in the same cell (Cao, Y. et al., (1996) J Biol Chem 271, 3154-62; DiSalvo, J. et al., (1995) J Biol Chem 270, 7717-23. Distribution studies show that these factors are often expressed in overlapping tissues and cells. Thus, PLGF/VEGF or VEGF/VEGF-B heterodimers are naturally present in tissues when both factors are synthesized in the same population of cells (Cao, Y. et al., supra; Cao, Y. et al., (1996) supra).

### SUMMARY OF THE INVENTION

The present invention provides a method for inhibiting the activity of VEGF (also referred to as VEGF-A) using gene therapy and, thus, for treating a variety of diseases caused by VEGF-induced angiogenesis. The method involves delivering a gene encoding a VEGF binding member, such as PLGF or VEGF-B, to a cell which expresses VEGF, such that the binding member forms a heterodimer with VEGF when the two proteins are co-expressed in the cell. As demonstrated by the studies described herein, heterodimers of VEGF/PLGF and VEGF/VEGF-B have reduced angiogenic activity compared to VEGF/VEGF homodimers and, thus, inhibit the angiogenic activity of VEGF.

In a particular embodiment of the invention, the gene encoding the VEGF binding member is contained within a vector suitable for gene delivery. Such vectors include, for example, adenoviral vectors, retroviral vectors, lentiviral vectors, vaccinia viral vectors, adeno-associated viral vectors, RNA vectors, liposomes, cationic lipids, and transposons. In a preferred embodiment, the gene is contained within a retroviral vector or a lentiviral vector. The gene can be also be delivered or co-administered with another anti-angiogenic agent or anti-cancer agent.

The method of the present invention can be used in vitro or ex vivo to inhibit angiogenesis and/or tumor growth. The method also can be used in vivo to treat a variety of diseases involving VEGF-induced angiogenesis in subjects including animals and humans. Such diseases include, for example, a variety of cancers, diabetic retinopathy and autoimmune diseases, such as rheumatoid arthritis.

### BRIEF DESCRIPTION OF FIGURES

FIG. 1 compares the angiogenic activity of homo- and hetero-dimeric forms of PLGF and VEGF in vitro and in vivo. Panel (a) is a graph comparing cell migration in a Boyden chamber. Panel (b) compares corneal neovascularization induced by growth factors as seen under a stereomicroscope. Panel (c) is a graph comparing neovascularization of cells in presence or absence of growth factors.

FIG. 2 compares the chemotactic activity of VEGFR-2(+) PAE cells (Panel (a)) verses VEGFR-1 (+) PAE cells (Panel (b)) as measured in presence of conditioned media from cells expressing various growth factors.

FIG. 3 compares growth and vessel density of tumors expressing PLGF verses those not expressing PLGF in vivo. Panel (a) compares the rate of tumor cell proliferation. Panel (b) compares tumor volume. Panel (c) compares blood vessel density.

### DETAILED DESCRIPTION OF THE INVENTION

The present invention is based on the discovery that PLGF and related growth factors (e.g., that bind to the VEGR-1 receptor) act as a natural antagonist of VEGF (i.e., VEGF-A) when both factors are produced in the same population of cells, and that the underlying mechanism is due to the formation of VEGF heterodimers having reduced activity (e.g., compared to VEGF homodimers). Accordingly, the present invention provides a method for inhibiting VEGF activity, including VEGF-induced angiogenesis (e.g., in tumors), using gene delivery of a VEGF binding member other than VEGF itself, such as PLGF, in cells expressing VEGF.

As described herein, the invention shall be defined using the following terms and phrases:

The term “angiogenesis” refers to the generation of new blood supply, e.g., blood capillaries, vessels, and veins, from existing blood vessel tissue (e.g., vasculature). The process of angiogenesis can involve a number of tissue cell types including, for example, endothelial cells which form a single cell layer lining of all blood vessels and are involved with regulating exchanges between the bloodstream and the surrounding tissues. New blood vessels (angiogenesis) can develop from the walls of existing small vessels by the outgrowth of endothelial cells. Angiogenesis is also involved in tumor growth as it provides tumors with blood supply necessary for tumor cell survival and proliferation (growth).

The term “inhibiting angiogenesis” as used herein refers to complete or partial inhibition of angiogensis.

The term “gene” as used herein refers to DNA or RNA encoding a protein of interest, such as PLGF or VEGF-B. Genes encoding VEGF binding members used in the present invention are typically contained within an expression vector along with genetic elements necessary for expression of the gene by a cell. Such elements are well known in the art and include, for example, suitable promoters and enhancers.

The term “VEGF binding member” refers to a protein or peptide other than VEGF which bind to VEGF (also referred to as “VEGF-A”) and inhibit VEGF activity (e.g., VEGF-induced angiogenesis) as measured by, for example, the numerous VEGF activity assays described herein. VEGF binding members include, for example, PLGF, VEGF-B, and other proteins which naturally bind to VEGF and, optionally, also to VEGFR-1 (as does VEGF).

The terms “PLGF” and “VEGF-B” refer to PLGF and VEGF-B growth factors as well as functionally equivalent analogs that bind to (form heterodimers with) VEGF and reduce the activity of VEGF. Functionally equivalent analogs include, for example, functionally equivalent peptides or homologues derived from PLGF and/or VEGF-B that retain the ability to bind to VEGF and to reduce its activity compared to cells in which the PLGF, VEGF-B or analog thereof has not been delivered.

The term “expressed or administered at sufficient levels” in reference to VEGF binding members (e.g., PLGF and VEGF-B) refers to levels necessary to partially or fully inhibit VEGF activity (e.g., VEGF-induced angiogenesis). The VEGF binding member is preferably expressed at levels which are equal (e.g., a 1:1 ratio) or, more preferably, which are greater than the level of endogenous VEGF expressed within the cell, so that VEGF/PLGF heterodimers are formed within the cell at greater levels than VEGF/VEGF homodimers. For example, the VEGF binding member can be expressed at a ratio of 1:2. 1:3, 1:4, 1:5, 1:6, 1:7 or higher with respect to the level of VEGF expressed in the cell. This level of expression reduces the overall activity of VEGF that would occur in the absence of expressing the VEGF binding member and is referred to as “over-expression” of the VEGF binding member. Moreover, in some cases (depending on the cells being treated), the VEGF binding member may already expressed naturally (endogenously) within the cell, such that delivery of the gene encoding the VEGF binding member to the cell increases the overall level of VEGF binding member expression to a level which reduces or blocks VEGF activity.

Assays for measuring or quantifying protein levels (e.g., standard ELISA assays) of VEGF binding member compared to VEGF, and for measuring VEGF activity are well known in the art and include, for example, those described in the studies provided herein.

The term “retroviral vector” refers to a vector containing structural and functional genetic elements that are primarily derived from a retrovirus, such as type c retroviruses. Suitable retroviral vectors include, for example, Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). “Retroviral vectors” used in the invention can also include vectors derived from human T cell leukemia viruses, HTLV-1 and HTLV-2, and the lentiviral family of retroviruses, such as human Immunodeficiency viruses, HIV-1, HIV-2, simian immunodeficiency virus (SIV), feline immonodeficiency virus (FIV), equine immunodeficiency virus (EIV), and other classes of retroviruses.

“Retroviruses” are RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles. At each end of the provirus are structures called “long terminal repeats” or “LTRs.” The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions, appears at both the both the 5′ and 3′ ends of the viral genome.

The term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

The term “vector” refers to a nucleic acid molecule capable of transporting (e.g., into a cell) another nucleic acid to which it has been linked. The term “expression vector” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions.

The term “gene delivery” or “transfection” refers to the introduction of exogenous DNA or RNA into eukaryotic cells. Gene delivery in the present invention can be accomplished in vitro, in vivo and ex vivo using any of a variety of means well known in the art. For example, in vitro and ex vivo gene delivery can be accomplished using techniques such as calcium phosphate-DNA co-precipitation, DEAE-dextran-mediated transfection, polybrene-mediated transfection, electroporation, microinjection, liposome fusion, lipofection, protoplast fusion, biolistics and viral transduction. In vivo gene delivery can be achieved using a variety of art-recognized techniques including, most commonly, injection (e.g., intravenous, intramuscular etc.).

In a preferred embodiment of the invention, the gene is delivered within a viral vector, preferably within a retroviral or lentiviral vector. A particular lentiviral vector which can be used in the present invention includes the self-activating lentiviral vector described in U.S. Provisional Patent Application Ser. No. 60/288,042, the contents of which are incorporated by reference herein.

Vectors (e.g., retroviral and lentiviral vectors) used in the present invention for gene delivery also can be incorporated into virions using packaging cell lines prior to contact with a cell, as is well known in the art. The phrase “packaging cell line” refers to a cell line (typically a mammalian cell line) which contains the necessary coding sequences to produce viral particles which lack the ability to package DNA or RNA and produce replication-competent helper-virus. When the packaging function is provided within the cell line (e.g., in trans by way of a plasmid vector), the packaging cell line produces recombinant virus, thereby becoming a “producer cell line.” Any suitable packaging cell line can be used in the present invention depending on the nature of the vector. Preferred packaging cell lines include retroviral and lentiviral packaging cell lines, such as the cell line described in PCT/US99/10585, the contents of which are hereby incorporated by reference.

In addition, the gene encoding the VEGF binding members, such as PLGF and VEGF-B, can be delivered either alone or in combination with one or more other angiogenesis-inhibiting (“anti-angiogenic”) factor(s), such as endostatin or angiostatin (see e.g., U.S. Pat. Nos. 6,174,861 and 6,024,688, the contents of which are incorporated herein by reference)), or one or more anti-cancer agents, such as chemotherapeutic agents or radiation. Moreover, multiple genes encoding different VEGF binding members can be concurrently delivered to enhance VEGF inhibition.

Accordingly, the present invention provides an improved method for treating diseases caused by VEGF activity and VEGF-induced angiogenesis (e.g., cancer, diabetic retinopathy, and rheumatoid arthritis) using gene therapy and a variety of gene delivery systems, such as retroviral and lentiviral gene delivery systems. Such systems provide the advantage of sustained, high-level expression of transferred therapeutic genes in vivo, and are highly efficient at infecting and integrating in a non-toxic manner into the genome of a wide variety of cell types. The systems also can be pseudotyped with an envelope protein, such as the vesicular stomatitis virus G-protein (VSV-G), using techniques known in the art (see e.g., Chesebro et al. (1990) J Virol 64 (1): 215-221; Naldini et al. (1996) Science 272: 263; U.S. Pat. No. 5,665,577 (Sodroski et al.); and WO 97/17457 (Salk Institute) to increase their target cell range.

### Equivalents

Although the invention has been described with reference to its preferred embodiments, other embodiments can achieve the same results. Those skilled in the art will recognize or be able to ascertain using no more than routine experimentation, numerous equivalents to the specific embodiments described herein. Such equivalents are considered to be within the scope of this invention and are encompassed by the following claims.

### Incorporation by Reference

The contents of all references and patents cited herein are hereby incorporated by reference in their entirety.

### EXAMPLES

Materials and Methods

Retroviral Design and Production

Complementary cDNAs coding for human PLGF₁₂₉ and VEGF₁₆₅ were cloned into the Murine Stem Cell Virus (MSCV) vector containing the enhanced green fluorescent protein (EGFP) (Clontech, Palo Alto, Calif.) fused 3′ of the Internal Ribosomal Entry Site from the Encaphalomyocarditus Virus (Novagen, Madison, Wis.) and 5′ of the Woodchuck Post-Transcriptional Regulatory Element (WPRE). Retroviral supernatants were generated by transfecting retroviral constructs into 293T cells along with expression plasmids encoding ecotropic gag/pol and the Vesicular Stomatitis Virus-Glycoprotein (VSV-G) envelope using a classical CaPO4 transfection method. The absence of Replication Competent Retrovirus (RCR) was verified by the inability to serially transfer viruses conferring G418 resistance to NIH3T3 cells.

Tumor Cell Transduction and Ex Vivo Selection

Tumor cell transduction and ex vivo selection was performed in murine T241 fibrosarcoma cells grown in log phase. The cells were exposed to filtered viral supernatants in the presence of 8 μg/ml of protamine sulfate on Retronectin™ (Biowhittaker, East Rutherford, N.J.) coated culture dishes for 6 hours on two consecutive days. EGFP positive cells were sorted using a FACStar+(Becton Dickinson, San Jose, Calif.) equipped with a 5-W argon and 30-mW neon laser. The retrovirally-transduced cells were analyzed for GFP cDNA by PCR and Southern blot analyses using standard methods.

Chemotaxis Assay

The motility response of VEGFR-expressing Porcine Aorta Endothelial (PAE) cells to various growth factors and conditioned media was assayed by a modified Boyden chamber technique using micropore nitrocellulose filters (8 μm thick, 8 μm pores). Cells were trypsinized and resuspended at 0.8×106 cells/ml in serum-free medium containing 0.2% BSA. The cells (40,000 cells per well) were placed in the upper chamber in serum-free medium containing 0.2% BSA with or without 50 ng/ml of VEGF, PLGF, PLGF/VEGF heterodimers or 25% conditioned media from different retrovirus-transduced cells in the lower chambers. After 4 h at 37° C., the medium was removed and cells attached to the filter were fixed in methanol and stained with Giemsa solution. All experiments were performed in triplicates. The cells that had migrated through the filter were counted and plotted as number of migrating cells per optic field (×32). The results demonstrate that VEGF homodimers exhibited a strong motility effect on VEGFR-2/PAE cells. In contrast, PLGF homodimers and PLGF/VEGF heterodimers were unable to induce migration of these endothelial cells.

Cell Shape Assay and Actin Staining

VEGFR-1/PAE and VEGFR-2/PAE cells were grown on coverslips in 12-well plates to about 40-60% confluency in Ham's F12 medium supplemented with 10% FCS. The medium was removed and replaced with fresh Ham's F12 medium containing 2% FCS with or without 100 ng/ml of VEGF, PLGF, PLGF/VEGF, or 25% of conditioned media. After 16 h, cells were fixed with 3% paraformaldehyde in PBS (pH 7.5) for 30 min, rinsed three times with PBS, and permeabilized with 0.5% Triton X-100 in PBS for 15 min. The cells were then washed three times with PBS and stained for 30 min with 1 ug/ml of TRITC-phalloidin (Sigma) in PBS. After washing 3 times with PBS, the coverslips were mounted in a mixture of glycerol and PBS (9:1) and the cells were examined in a combined light and fluorescence microscope.

### Example 1

### Expression of PLGF in T241 Fibrosarcoma Cells Results in Formation of PLGF/VEGF Heterodimers and Inhibits Formation of VEGF Homodimers

The ability of PLGF to heterodimerize with VEGF in tumor cells was tested by expressing PLGF to a high level from a retroviral vector in a well-characterized murine fibrosarcoma cell line which expresses mouse VEGF (mVEGF) and whose growth is VEGF-dependent. Retroviruses expressing human PLGF (hPLGF) or human VEGF (hVEGF) were used to infect T241 cells and EGFP-positive cells (positive transfectants) were sorted by FACStar. wt T241 cells were used as a negative control.

The retrovirally transduced cells were analyzed for GFP cDNA by Southern blot analysis. hPLGF-T241, hVEGF-T241 or wt T241 cells were metabolically labeled with 35S-methionine and conditioned media were harvested after 16 h. Radiolabeled complexes of hPLGF homodimers and hPLGF/mVEGF heterodimers were immunoprecipitated with an anti-hPLGF antibody. Immunocomplexes of hVEGF homodimers and hVEGF/mVEGF heterodimers were precipitated with an anti-hVEGF antibody. The dimeric and monomeric forms of growth factors were analyzed on a SDS gel under non-reducing and reducing conditions.

Overexpression of hVEGF in murine fibrosarcoma cells resulted in the formation of homodimers of hVEGF/hVEGF and heterodimers of hVEGF/mVEGF that were co-precipitated with the specific antibody to hVEGF. Similarly, high expression of hPLGF caused the formation of hPLGF/hPLGF homodimers and hPLGF/mVEGF heterodimers as detected in complexes precipitated by a specific anti-hPLGF antibody. Again, both mVEGF₁₆₄ and mVEGF₁₂₁ were involved in the heterodimerization with HPLGF.

The amount of mouse VEGF homodimers secreted by wt T241 and hPLGF-T241 cells was also quantified using ELISA. A high level of mVEGF homodimers (1300 pg/ml) was detected in 72 h-conditioned medium derived from wt T241 tumor cells. In contrast, levels of mVEGF homodimers were non-detectable in the conditioned medium of hPLGF-T241 cells under the same conditions. These results indicate that overexpression of PLGF in fibrosarcoma cells inhibits the formation of VEGF homodimers required for VEGF activity by favoring the formation of PLGF/VEGF heterodimers having reduced VEGF activity. Thus, overexpression of PLGF in tumor cells can be used to inhibit VEGF activity.

### Example 2

### Comparison of the Angiogenic Activity of Homodimeric and Heterodimeric Forms of PLGF and VEGF

The angiogenic activity of purified homo- and heterodimeric forms of PLGF and VEGF proteins were assessed as described below. VEGF, PLGF, and PLGF/VEGF were purified to homogeneity, analyzed in a SDS gel under reducing and non-reducing conditions, followed by staining with Coomassie blue.

I. Chemotactic Activity

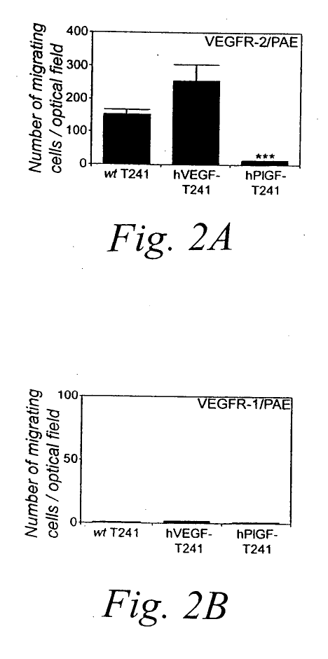

The chemotactic activity of growth factors in mono and dimeric forms was tested by studying the effect of conditioned media derived from various transduced and non-transduced cells in a Boyden chamber assay as described in the Methods section above. As shown in FIG. 2, the conditioned medium of wt T241 cells significantly stimulated VEGFR-2/PAE cell migration. High expression of hVEGF enhanced the chemotactic activity produced by T241 cells. In contrast, expression of PLGF completely abolished the chemotactic effect produced by T241 cells. None of these conditioned media significantly induced VEGFR-1/PAE cell motility. These data demonstrate that overexpression of PLGF in T241 cells completely neutralizes tumor-produced angiogenic motile activity.

II. Cell Shape Changes and Actin Reorganization

To assess the ability of growth factors to cause cell shape changes and actin reorganization in tumor cells, cytoskeletal analysis and actin staining were performed as described in the Methods section above. As observed by microscopy, VEGF homodimers induced dramatic spindle-like cell shape change with reorganization of actin fibers. In contrast, both PLGF homodimers and PLGF/VEGF heterodimers failed to induce this morphological change. Furthermore, VEGFR-1-expressing PAE cells did not respond to any of these three factor-treatments.

Specifically, incubation of conditioned medium derived from wt T241 cells resulted in an elongated spindle cell shape change in VEGFR-2/PAE cells, similar to that induced by hVEGF, confirming that T241 cells secrete high levels of mVEGF. This effect was completely abrogated after expression of hPLGF in these cells, indicating that a majority of the mVEGF molecules participated in formation of heterodimers with hPLGF. In contrast, overexpression of hVEGF in T241 cells led to remarkable cell shape changes and actin reorganization.

Collectively, the data presented above demonstrate that overexpression of PLGF antagonizes the angiogenic activity of VEGF produced by tumors.

### Example 3

### Expression of PLGF Inhibits Angiogenesis in a Mouse Corneal Micropocket Assay

The ability of growth factors to induce angiogenesis in vivo was also investigated using the mouse cornea model. Corneal micropockets were created with a modified von Graefe cataract knife in both eyes of each male 5-6-wk-old C57B16/J mouse. A micropellet (0.35×0.35 mm) of sucrose aluminum sulfate (Bukh Meditec, Copenhagen, Denmark) coated with hydron polymer type NCC (IFN Sciences, New Brunswick, N.J.) containing 160 ng of PLGF, VEGF, or PLGF/VEGF was implanted into each corneal pocket. The pellet was positioned 0.6-0.8 mm from the corneal limbus. After implantation, erythromycin/ophthalmic ointment was applied to each eye. Eyes were examined by a slit-lamp biomicroscope on day 5 after pellet implantation. Vessel length and clock hours of circumferential neovascularization were measured.

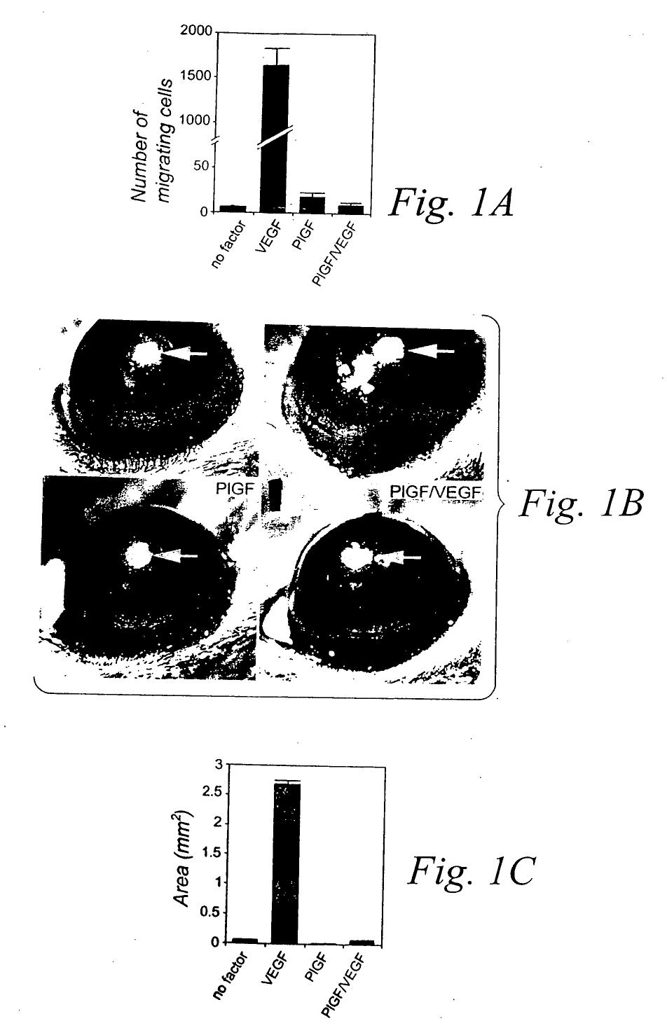

As shown in FIG. 1, VEGF homodimers induced a strong angiogenic response with the formation of a high number of microvessels that form primitive vascular network plexuses at the leading edge. In contrast, the same amount of PLGF homodimers or PLGF/VEGF heterodimers failed to stimulate corneal angiogenesis. These data also show that the molecular mechanism of the anti-VEGF effect by PLGF is due to the formation of functionally inactive PLGFNEGF heterodimers.

### Example 4

### Expression of PLGF Suppresses Tumor Growth In Vivo

To study if hPLGF-expressing cells could affect tumor growth in vivo, wt T241, hPLGF-T241 or hVEGF-T241 tumor cells were subcutaneously implanted into syngeneic C57B16/J mice. Tumor sizes were measured every other day. Tumor volumes were calculated according to the formula width²×length×0.52. At day 14 after tumor implantation, typical tumor appearance was photographed. The growth of metastases in hVEGF-T241-implanted mice and the implanted tumors were further characterized. At day 18 after tumor implantation, primary tumors and livers were resected and stained with an anfi-CD31 antibody or stained with haemotoxylen-eosin alone at 10× magnification. Microvessels in tumors were revealed by immunoperoxidase, followed by development with DAB substrate. Microvessel density was counted under a light microscope in at least 5 random fields (g). *** P<0.001.

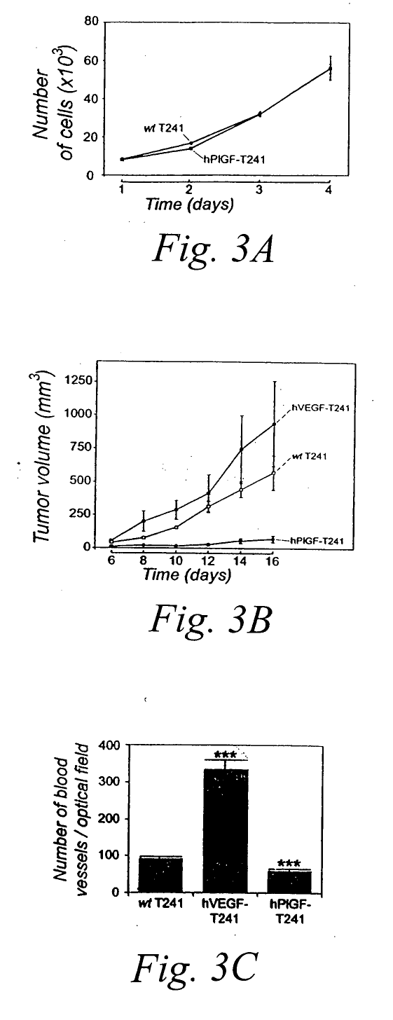

As shown in FIG. 3, wt T241 tumor cells grew rapidly in vivo, and visible tumors were readily detectable by 5 days after implantation and grew exponentially after day 8 of implantation. Expression of hVEGF in these cells significantly accelerated tumor growth, but not tumor formation, indicating that tumor growth, but not the initial take-off, is dependent on the process of angiogenesis. Tumors expressing hVEGF grew invasively into surrounding tissues and resulted in early metastases in other organs such as spine and liver. In contrast, expression of hPLGF remarkably delayed tumor take-off (formation) and visible tumors were only detectable by day 12 after implantation. These tumors remained at a similar small average size of 70 mm³ by 16 days after implantation. At day 16 of tumor implantation, approximately 90% inhibition of tumor growth was scored in hPLGF-expressing tumors as compared with the wt T241 tumors.

These results show that expression of PLGF in VEGF-producing tumors causes formation of functionally inactive PLGF/VEGF heterodimers and thus neutralizes VEGF activity. Furthermore, these results show that the molecular mechanism of the anti-VEGF effect by PLGF is due to the formation of functionally inactive PLGF/VEGF heterodimers when the two factors are simultaneously synthesized intracellularly in the same population of cells.

What is claimed is:

1. A method of inhibiting angiogenesis comprising delivering a gene encoding a VEGF binding member selected from the group consisting of PLGF and VEGF-B to a cell which expresses VEGF, such that the binding member is co-expressed with VEGF in the cell.

2. The method of claim 1, wherein the VEGF binding member is PLGF.

3. The method of claim 1, wherein the VEGF binding member forms a heterodimer with VEGF within the cell.

4. The method of claim 1, wherein the gene is contained within a vector.

5. The method of claim 4, wherein vector is selected from the group consisting of an adenoviral vector, a retroviral vector, a vaccinia virus, an adeno-associated viral vector, an RNA vector, a liposome, a cationic lipid, a lentiviral vector, an Adeno-associated Virus, and a transposon.

6. The method of claim 1, wherein the gene is co-administered with another anti-angiogenic agent.

7. The method of claim 1, wherein the cell expressing VEGF is a tumor cell.

8. The method of claim 1, wherein the gene is delivered to a subject suffering from a disease caused by angiogenesis.

9. The method of claim 8, wherein the disease is cancer.

10. The method of claim 8, wherein the disease is diabetic retinopathy.

11. The method of claim 8, wherein the disease is rheumatoid arthritis.
